(19) United States
(12) Patent Application Publication
(10) Pub. No.: US 2018/0104336 A1
(43) Pub. Date: Apr. 19, 2018

(54) Method of Increasing Radiation Sensitivity of Tumor Cells

(71) Applicants: Gunnar Boysen, Little Rock, AR (US); Robert J. Griffin, Little Rock, AR (US)

(72) Inventors: Gunnar Boysen, Little Rock, AR (US); Robert J. Griffin, Little Rock, AR (US)

(21) Appl. No.: 15/783,427

(22) Filed: Oct. 13, 2017

Related U.S. Application Data
(60) Provisional application No. 62/407,643, filed on Oct. 13, 2016.

(51) Int. Cl. A61K 41/00; A61K 31/501; A61K 31/433; A61N 5/10

(52) U.S. Cl. CPC A61K 41/0038; A61K 31/501; A61N 2005/1098; A61N 5/10; A61K 31/433

(57) ABSTRACT

A method of increasing radiation sensitivity of tumor cells in a subject is disclosed. The method includes the step of administering to the subject an effective amount of a glutaminase inhibitor, such as CB-839 or BPTES. A method of treating a tumor in a subject is also disclosed, which includes the steps of administering to the subject an effective amount of a glutaminase inhibitor and exposing the tumor to radiation in a dose effective to reduce a size of the tumor.

### CROSS-REFERENCE TO RELATED APPLICATIONS

This application claims the benefit of U.S. Provisional Application No. 62/407,643, entitled “Method of Increasing Radiation Sensitivity of Tumor Cells” and filed on Oct. 13, 2016. The complete disclosure of said patent application is hereby incorporated by reference.

### STATEMENT REGARDING FEDERALLY SPONSORED RESEARCH OR DEVELOPMENT

This invention was made with government support from grant nos. UL1TR000039 and KL2TR000063 awarded by the National Institute of Health (NIH) Clinical and Translational Science Award (CTSA) program. The government has certain rights in the invention.

### BACKGROUND OF THE INVENTION

Lung cancer is the leading cause of cancer-related death in the U.S. with an expected 228,000 new cases and 160,000 deaths per year. Despite immense research efforts, the overall 5-year survival rate of less than (<) 17% remains low compared to other cancers. In the past, treatment of advanced lung cancer followed a straightforward algorithm of platinum-based combination therapies or third-generation cytotoxic drugs, irrespective of histopathology subtypes. More recently, treatment efficacies have improved due to pre-selection based on histopathology sub-types and identification of specific driver mutations. Including a patient's tumor biology in therapy selection (personalized medicine) is transforming the diagnosis and treatment of lung cancer. The inventors recently studied metabolite profiles in lymph node aspirates containing malignant lung tumor cells and found a strong correlation between glutamine consumption and glutathione excretion (Sappington et al. 2017).

Mechanistic studies in tissue culture and animal models suggest glutamine utilization and glutathione synthesis are important in cancer promotion and progression (Sappington et al. 2016). The main explanation is that glutamine provides additional carbon and nitrogen sources for cell growth. In parallel, tumor cells are known to increase glutathione synthesis, and intracellular glutathione concentrations in tumor cells are reported to be as high as 10 mM. Glutathione, the most abundant thiol-compound: (i) functions as an antioxidant, (ii) is a precursor for conjugation reactions, (iii) participates in maintenance of the cysteine pool and (iv) contributes to the regulation of cellular processes, including apoptosis (Boysen 2017).

Glutamine is an essential nutrient and the most abundant free amino acid in human serum. The first step in utilization of glutamine is its conversion to glutamate by glutaminase (GLS). Glutamate dehydrogenase converts glutamate to alpha-ketoglutarante, an important tricarboxylic acid cycle metabolite. The glutamine dependence of tumor cells is known and GLS inhibitors are under development. Glutamate is a precursor for glutathione synthesis, which is carried out by two conjugation reactions catalyzed by glutamate cysteine ligase and glutathione synthase (Vander Heiden, Cantley, and Thompson 2009). Therefore, it is conceivable that glutamine-derived glutamate might also contribute to cellular defense by supplying necessary glutamate for glutathione synthesis.

To test this hypothesis, several lung tumor cell lines were selected and their glutamine consumption and glutathione synthesis and excretion patterns were established. The importance of glutamine-derived glutamate for glutathione synthesis and cell proliferation is demonstrated by: (i) culturing in glutamine-free media, (ii) utilization of stable isotope-labeled [¹³C₅]-glutamine, (iii) inhibition of GLS by Bis-2-(5-phenyl-acetamido-1,3,4-thiadiazol-2-yl) ethyl sulfide (BPTES), a known GLS inhibitor (Shukla et al. 2012) and (iv) the presence of glutathione-ester, the bioavailable form of glutathione, prevents BPTES-induce cytotoxicity. Sensitivity to radiation treatment demonstrated the role of glutamine-derived glutathione in radiotherapy resistance. Lastly, in vivo studies show that GLS-inhibitor, CB-839, increases response to radiation therapy.

These and other features, objects and advantages of the present invention will become better understood from a consideration of the following detailed description of the preferred embodiments and appended claims in conjunction with the drawings as described following:

### BRIEF SUMMARY OF THE INVENTION

The present invention is directed to a method of increasing radiation sensitivity of tumor cells and tumors of human patients. The inventors have data that demonstrate that glutathione is exclusively derived from glutamine. Glutathione is an endogenous antioxidant that protects cells from oxidative stress such as induced by radiation therapy. GLS inhibitors, such as BPTES and CB-938, have been shown to be effective radiosensitizers. In vitro studies demonstrate that CB-839 successfully depletes glutathione synthesis and excretion and led to increase sensitivity to ionizing radiation. In vivo, short term GLS-inhibitor, CB-839, treatment reduced serum glutathione by >50%. BPTES and CB-839 were used as model GLS-inhibitors to inhibit glutamine utilization to block glutamine dependent tumor growth. The inventors show that BPTES and CB-839 can be used to deplete glutathione and thereby improve response to radiation therapy. A short term CB-839 treatment (<3 days) is sufficient to deplete glutathione and sensitize tumors to radiation therapy. Usually CB-839 is given chronically twice a day for the duration of treatment (usually >30 days). Thus, combination therapy will be a more effective therapy, reduce side effects and lower costs.

Using the H460 lung tumor xenograft model, the inventors show that short term (3 times) CB-839 treatment, which alone does not show efficacy, significantly improves response to radiation treatment by more than 25% based on tumor volume measurements over time. CB-839 is in phase I clinical trials and given at relative high dose every 12 hrs of the duration of the therapy that in animals lasts more than (>) 30 days. GLS-inhibitors, such as CB-839, in combination with radiation therapy can be as or more effective in shirking of tumors and delaying tumor growth. It is shown that GLS-inhibitors can be as effective when given short-term in combination with radiation instead of daily dosing (chronic).

The mode of action is that GLS-inhibitors inhibits glutaminase and subsequently depletes glutathione. The initial finding is derived from tumor diagnosis research where metabolomes were used to determine the alterations in tumor cell metabolism. The inventors observed a strong mechanistic relationship between glutamine consumption and glutathione synthesis and excretion in about 30% of the lung cancer patients. Subsequent cell line studies confirm this mechanic link to be active in lung tumor cells. Use of GLS-inhibitors, such as BPTES and CB-839, in combination with radiation therapy can be applied to patient preselected based on their glutamine dependency and rate of glutathione synthesis. Therefore, the proposed re-purposing of GLS-inhibitors, such as BPTES and CB-839, as radiation sensitizer should be effective in 30% of all lung cancer patients. The fact that lung cancer patients suitable for this therapy can be prescreened tremendously increases response rates.

Glutamine is essential for synthesis and excretion of glutathione to promote cell growth and viability (FIG. 1). Increased glutamine uptake is known to drive cancer cell proliferation, making tumor cells glutamine-dependent. Glutamine provides additional carbon and nitrogen sources for cell growth. The first step in glutamine utilization is its conversion to glutamate by glutaminase (GLS). Glutamate is precursor for glutathione synthesis, and the inventors investigated the hypothesis that glutamine drives glutathione synthesis and thereby contributes to cellular defense systems. The importance of glutamine for glutathione syntheses was studied in H460 and A549 lung cancer cell lines using glutamine free culture and the GLS inhibitor BPTES. Metabolic activities were determined by targeted mass spectrometry. A significant correlation between glutamine consumption and glutathione excretion was demonstrated in H460 and A549 tumor cells (FIG. 2C). Culturing in in presence of [¹³C₅] glutamine demonstrated that, by 12 hrs, more than (>) 50% of excreted glutathione is derived from glutamine (FIG. 3). Culturing in glutamine-free media or treatment with glutaminase (GLS)-specific inhibitors, BPTES or CB-839, reduced cell proliferation and viability, and abolished glutathione excretion (FIG. 4 and FIG. 8). Treatment with glutathione-ester prevented BPTES induced cytotoxicity (FIG. 5). Inhibition of GLS markedly radiosensitized the lung tumor cell lines, suggesting an important role of glutamine-derived glutathione in determining radiation sensitivity (FIG. 6). The inventors demonstrated that a significant amount of extracellular glutathione is directly derived from glutamine. This finding adds yet another important function to the already known glutamine dependence of tumor cells and probably tumors as well.

### BRIEF DESCRIPTION OF THE DRAWINGS

FIG. 1 shows the metabolic scheme of the glutamine uptake, metabolism to glutamate and utilization for GSH synthesis. The upward arrows indicate metabolites increased in fast proliferating lung tumor cell lines. The downward arrows indicate metabolites consumed in fast proliferating lung tumor cell lines. Also shown are the glutaminase (GLS) enzyme and a GLS-inhibitor.

FIG. 2A-2B shows a time course of extracellular (FIG. 2A) glutamine and (FIG. 2B) glutathione in lung tumor cell lines. FIG. 2C shows the correlation of glutamine consumption to glutathione production in H460 and A549 cells, respectively. At the indicated time points, 25 μl of medium from cultured cells was analyzed. FIGS. 2D-2E show the intracellular concentrations of (FIG. 2D) glutamine, and (FIG. 2E) glutathione at 48 hrs. Solid columns indicate culturing in complete media and striped columns indicate culturing in glutamine-free media. Metabolite concentrations in media were significantly different between each other at each time point, except between A549 and MRC-5 at 24 hrs (p<0.01). Intracellular amounts were significantly lower in cells grown in glutamine-free media compared to cells grown in complete media (p<0.01). Some error bars are smaller than the symbol and therefore are not visible. FIG. 2F shows growth curves of cell lines in complete and glutamine-free media.

FIG. 3A-3B shows a time course of (FIG. 3A) [¹³C₅]-glutamate and (FIG. 3B) [¹³C₅]-glutathione derived from [¹³C₅]-glutamine in A549 and H460 cells. Cells were grown in glutamine-free medium supplemented with 2 mM [¹³C₅]-glutamine. At the indicated time points, 25 μl of culture medium was analyzed for the presence of stable isotope-enriched metabolites.

FIG. 4A-4D show the effect of GLS inhibition by BPTES on cell clonogenic viability (FIG. 4A), on cell viability using CellTiter-Glo® (FIG. 4B), cell toxicity (FIG. 4C) and glutathione excretion (FIG. 4D) in H460 and A549 cells. Shown are mean±SD of three independent experiments. FIG. 4E shows representative bright field images (40× magnification) of H460 and A549 cells after 48 hrs treatment with various concentrations of BPTES or culturing in glutamine-free (Gln-) media.

FIG. 5A-B show the protective effect of glutathione-ether (GSHE), the bioavailable form of glutathione, on BPTES-treated cells. Shown are the (FIG. 5A) viability and (FIG. 5B) cell toxicity responses. H460 and A549 cells were treated with 10 μM BPTES in presence of 1-10 mM GSHE. Shown are mean±SD of three independent experiments.

FIG. 6A-6B show the effect of culturing in glutamine-free media or 2 μM BPTES treatment on radiation response in (FIG. 6A) H460 and (FIG. 6B) A549 tumor cells. Radiation sensitivity was determined utilizing the clonogenic assay and normalizing to the plating efficiency in untreated cells. Shown are mean±SD of three independent experiments. Some error bars are smaller than the symbol and therefore are not visible.

FIG. 7A-7C show the concentrations of (FIG. 7A) extracellular glutamate, (FIG. 7B) intracellular glutamate, and (FIG. 7C) intracellular γ-glutamyl-cysteine at 48 hours. Solid columns indicate culturing in complete media and striped columns indicate culturing in glutamine-free media.

FIG. 8A-8B show the effect of GLS-inhibitor CB-839 on (FIG. 8A) clonogenic viability (x-axis is in [nM]) and (FIG. 8B) viability assessed by MMT assay.

FIG. 9A-9B show the (FIG. 9A) mean and (FIG. 9B) relative response of H460 mouse xenografts to radiation with and without CB-839 treatment in male nude mice. Vehicle; solid squares, 18 Gy radiation; solid circles, CB-839; open diamonds, CB-839 plus 18 Gy radiation; open triangle.

FIG. 10A-10F show the relative response of H460 mouse xenografts to various dosages of radiation with and without CB-839 treatment in female nude mice. FIG. 10A is a spaghetti plot for all groups. FIG. 10B shows a comparison of vehicle control and CB-839. FIGS. 10C-10F show a comparison of vehicle control to 2,4, 8, or 12 Gy radiation, respectively, with and without CB-839.

FIG. 11 shows the concentration of total glutathione in serum at day of radiation in female and male mice treated with vehicle or CB-839 28, 16 and 4 hrs prior to radiation. Blood was drawn from tail vain and serum was separated from red blood cells. Reduced and oxidized glutathione were measured by LC-MS and sumed to give total glutathione. Solid bars indicate male mice. Striped bars indicate female mice. Each bar represents the mean and SD from 5 mice each.

### DETAILED DESCRIPTION OF THE INVENTION

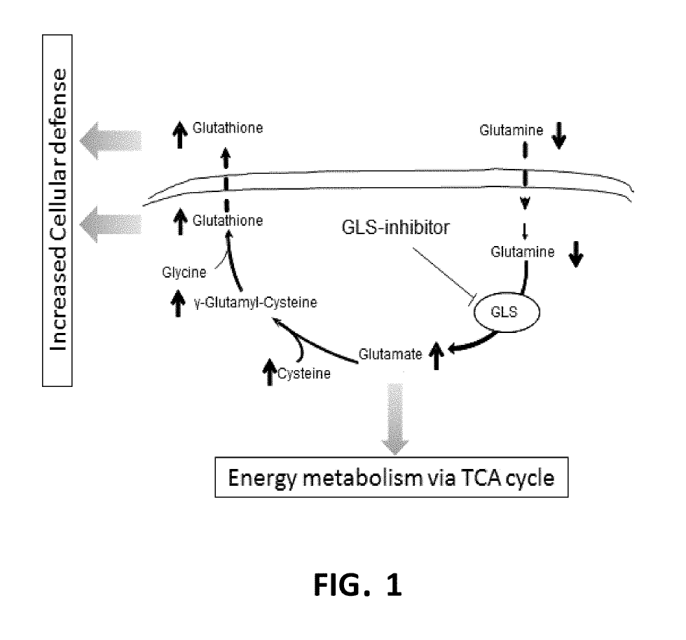

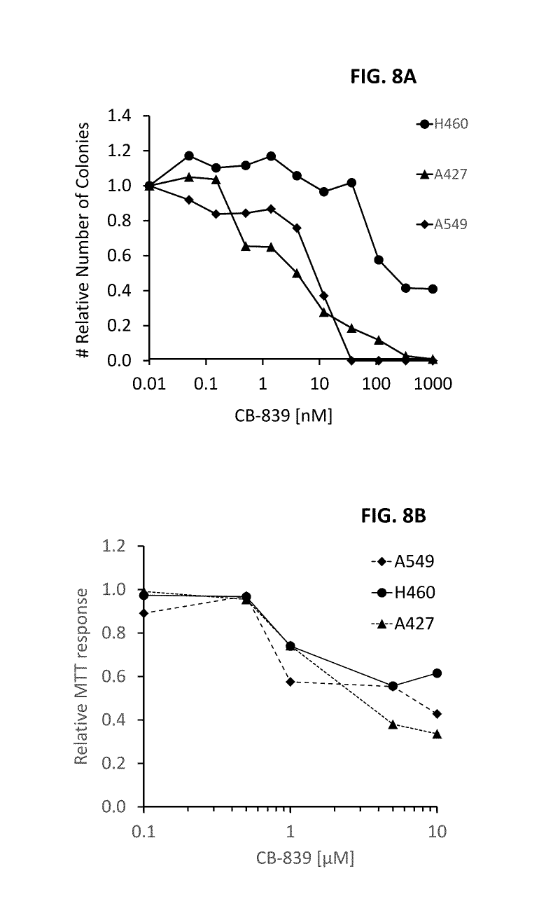

With reference to FIGS. 1-11, the preferred embodiments of the present invention may be described. The present invention is directed to a method of increasing radiation sensitivity of tumor cells. In vivo and in vitro studies demonstrating that GLS-inhibitors, such as CB-839 and BPTES, successfully depletes glutathione and results in increased sensitivity to ionizing radiation are explained below.

### In Vitro Study

MATERIALS AND METHODS: Cell culture: H427, H460 and A549 lung tumor and MRC-5 alveolar fibroblast cell lines were purchased from ATCC and cultured in standard incubation conditions using 89% RPMI Medium 1640+L-Glutamine (Sigma St. Louis, Mo.), 10% fetal bovine serum, and 1% penicillin-streptomycin (complete medium) at a humidified 37° C. with 5% CO₂. Each cell line was propagated from an initial concentration of 100,000 cells per flask. Sub-cultures of each cell line were then seeded into 25-T flasks at 100,000 cells/flask. Cells were allowed to attach for 24 hours.

Glutamine-free medium experiments: Media were removed from all flasks (Time 0 hrs) and 5 replicates from each line were given 5 mL complete medium and 5 replicates were given 5 mL glutamine-free complete medium (Sigma St. Louis, Mo.). Media aliquots of 100 μl were removed from each flask at 12 hour intervals to monitor the metabolomic footprint. After 48 or 84 hours, media were removed, cells were washed with water (5 sec), and immediately flash frozen by the addition of 15 ml liquid nitrogen into the culture flasks and stored at −80° C. as describe previously.

GLS inhibition experiments: In the mechanistic experiments with BPTES or CB-839, cells (7500) were seeded (Time—24 hrs) into 96-well plates containing 100 μl complete medium, and allowed to attach for 24 hrs. Complete medium was added (Time 0 hrs) containing various concentrations of GLS inhibitors to give the final concentrations of 0.001 to 100 μM. Media aliquots of 25 μl were removed from each well at 24, 36, and 48 hrs to monitor the time course of metabolite consumption and production (metabolomic footprint). Cells were maintained at 37° C., 5% CO₂ and 40% humidity until time of harvest. Cell viability was determined using Cell Glow, Cell TOX or MTT assays according to manufacture descriptions.

Colognenic assay, Colognenic assay was carried out as described previously (Franken et al. 2006). Cells were seed in 6-well and allowed to attach for 1 day. Then media was replaced with media containing GLS-inhibitor (BPTES or CB-839) at various concentrations (e.g., 1-100 μM). Cells were incubated in a CO₂ incubator at 37° C. for 1- to 14 days until cells in control plates had formed visible colonies Cell were fixed and stained prior to counting of the colonies.

[¹³C₅]-glutamine experiments: To determine the fraction of glutathione that is derived directly from glutamine, A549 and H460 cell lines were cultured in 96-well plates in glutamine-free medium supplemented with 0.3 g/L [¹³C₅]-glutamine. Cell media aliquots were harvested at 12, 24, 36, and 48 hrs. Light microscopy was used at 12 hours to visualize the cells at each dosage. After 48 hours the CellTiter 96, CellTiter-Glo® 2.0, and CellTox™ Green Cytotoxicity assays, (Promega, Madison, Wis.) were utilized to respectively assess cell proliferation, cell viability, and cell death of both the BPTES and [¹³C₅]-glutamine experiments.

Glutathione-monoethyl ester (GSHE) experiments: Cells (7500) were seeded into 96 well plates containing 100 μl complete media. Cells were treated with 10 μM BPTES in DMSO and 0, 1, 5 and 10 mM GSHE. After 48 hrs cells were harvested and cell viability and toxicity were assessed as described above.

LC-MS quantitation by liquid chromatography tandem mass spectrometry (LC-MS/MS): Metabolites were extracted from cell pellets by the addition of 3 ml of 50% methanol/0.2% formic acid to the frozen cells in the culture flasks. Cells were scraped from the culture flasks and transferred to 15 ml tubes. Proteins were precipitated from 750 μl cell pellet suspensions by addition of 1050 μl acetonitrile/0.2% formic acid. Metabolites were extracted from 25 or 100 μl culture media from 96 well plates or 25-T flasks, respectively, and with the respective addition of 275 μl or 1300 μl of 40% methanol/60% acetonitrile/0.2% formic acid. Cell pellets or media suspensions were incubated on ice for 30 min, followed by centrifugation for 10 min at 13000 g. Supernatants were transferred to new vials, solvents were removed in a speed vac and the concentrated metabolites stored at -80° C. until analysis.

Samples were reconstituted in 0.2% formic acid and analyzed by LC-MS/MS (Agilent, 1290 Infinity LC coupled to an Agilent 6490 triple quadrupole mass analyzer). An Agilent Poroshell 2.7 μm C18 (2.1 mm×100 mm) column was operated with a linear gradient from 2% Methanol/0.01% formic acid to 95% Methanol/0.01% formic acid in 10 min. Individual metabolites were monitored in multiple reactions monitoring mode, monitoring specific ion transitions shown in Table 1. The specific ion transitions and retention times for each metabolite were established experimentally using commercially available standard compounds. Quantitation of the individual metabolites were based on external calibration curves that were generated with each set of samples. In the stable isotope tracing experiment, ¹³C₅-incorporation into subsequent metabolites was determined by monitoring the corresponding m/z ion transitions (Table 1).

TABLE 1
Summary of metabolites measured in lung
tumor cell lines and culture media.

| Metabolite | ¹²C | # ¹³C | ¹³C-incorporation |
|---|---|---|---|
| γ-Glutamyl-cysteine | 251.2 → 84.1 | 5 | 256.0 → 88.0 |
| Glutamate | 148.0 → 84.0 | 5 | 153.0 → 88.0 |
| Glutamine | 147.0 → 84.0 | 5 | 152.0 → 88.0 |
| GSH (reduced) | 308.0 → 84.0 | 5 | 313.0 → 88.0 |
| GSSG (oxidized)^{a} | 613.0 → 355.0 | 5 | 618.0 → 363.0 |
| | | 10 | 623.0 → 363.0 |

^{a}For the stable isotope tracing experiment, GSSG was acquired as m/z +5 and +10 precursor ions to monitor single or double incorporation of the ¹³C₅-carbon backbone.

Radiation exposure of lung tumor cell lines: Radiation sensitivity was determined by methods described previously. In brief, for colony formation, A549 and H460 cells were seeded in 6-well plates containing 3 ml standard complete medium and allowed to attach for 24 h. Media were changed and cells were grown for another 24 h in standard medium, glutamine-free medium, medium containing 2 μM BPTES (to deplete glutathione), and medium containing only the vehicle. Cells were subsequently radiated using a Faxitron X-ray Generating System (CP-160, Faxitron X-Ray Corp., Wheeling, Ill.). Single-doses of 4 or 8 Gy (Gray) were delivered at a dose rate of 1 Gy/min (150 kVp and 6.6 mA). Cells were then placed in standard complete RPMI medium and allowed to form colonies for 8 days. The surviving colonies (containing >˜50 cells) were stained and counted on a stereomicroscope. Plating efficiency (PE) of cells with each treatment were determined and normalized to that of control untreated cells and the surviving fractions were calculated by dividing the PE of the treated cells by the PE of the control untreated cells.

Statistical Analysis: Media and cell pellets were analyzed for differences between cell lines and glutamine treatment and the surviving fractions of cell colonies were compared among each cell line via Conover's Kruskal-Wallis method. The time-dependent relationships between glutamine consumption and glutathione production were analyzed by linear regression on each cell line. The resulting regression slopes (with their standard errors) were interpreted as “glutathione production ratios,” i.e., μM glutathione produced per mM glutamine consumed. Glutathione production ratios were compared for differences between cell lines via t-test using the combined standard errors of the ratios. All post-hoc comparisons employed a P<0.05 significance level despite the multiple comparisons, in order not to inflate Type II error in this study.

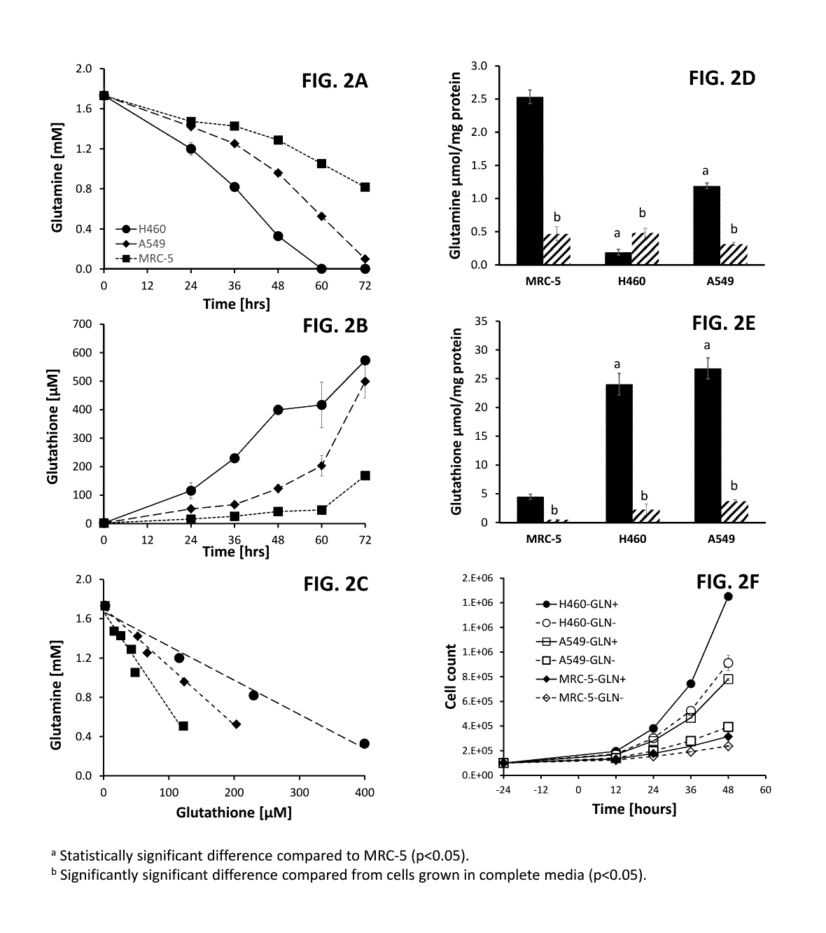

RESULTS: The two human lung carcinoma cell lines (H460 and A549) and one human alveolar fibroblasts (MRC-5) were grown in complete or glutamine-free media (FIG. 2F). Their doubling times, calculated based on propagation over 48 hours in complete media, had means ±standard deviations (SDs) of 12.4±0.02, 16.2±0.08 and 29.0±0.61 hours for H460, A549 and MRC-5, respectively. The doubling times for H460 and A549 are reported as 17.8 and 22.9 hours, suggesting that the cells grow slightly faster in the complete medium chosen for this study than in the recommended F12-K media. In glutamine-free media, cell growth was reduced and the doubling times had means±SDs of 15.1±0.43, 24.3±0.36 and 38.4±0.84 hours (H460, A549 and MRC-5, respectively). Of these, A549 was the most glutamine dependent, with a 33% growth reduction in glutamine-free medium compared to 17% and 23% growth inhibition of H460 and MRC-5, respectively (FIG. 2F). The different cell lines showed distinct variability in size and morphology (data not shown).

To study the potential mechanistic link between glutamine uptake and glutathione synthesis, the time courses for glutamine consumption and glutathione excretion were established in H460, A549 and MRC-5 cell lines (FIGS. 2A and 2B). The fastest growing H460 cell line was the most efficient in glutamine uptake, while the slower growing A549 and MRC-5 lines used much less glutamine during the same time period. For glutamine uptake all pairwise comparisons were statistically significant except between A549 and MRC-5 at 24 hrs. Likewise, the H460 cell line was the most efficient in glutathione synthesis, and MRC-5 the least efficient (FIG. 2B). All pairwise comparisons at each time point for glutathione were statistically significant. There was a statistically significant correlation between glutamine consumption and glutathione excretion (FIG. 2C). Regression analysis revealed glutathione production ratios±standard errors (in μM/mM of glutamine) of 278±19.9 for H460, 166±12.0 for A549, and 95±6.7 for MRC-5, all of which were statistically significantly different from each other.

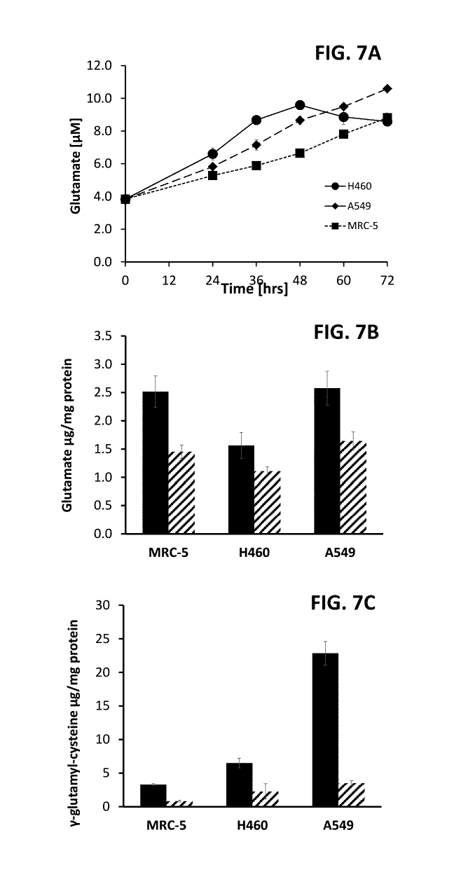

From the time course experiment, 48 hrs was chosen for subsequent studies, because glutamine is essentially depleted at 60 hrs in the fast growing H460 cell line. The excretion of glutamate increased in all cell lines over time (FIG. 7A). Interestingly, H460 cells start consuming glutamate after 48 hrs, coinciding with the depletion of glutamine. All cell lines receiving complete media showed significant differences in intracellular glutamine (FIG. 2D), glutamate (FIG. 7B), γ-glutamyl-cysteine (FIG. 7C), and glutathione (FIG. 2E) per mg protein as compared to glutamine-free complete media. Intracellular glutathione per mg protein were significantly higher (about 5-fold) in the carcinomas H460 and A549 cells than in MRC-5 fibroblasts, although no significant differences were observed between the tumor cell lines. This was true for the complete and glutamine-free media cultures. In presence of glutamine, the amounts of intracellular glutamate were not different between A549 and MRC-5 cells (FIG. 7B). Glutamate amounts were ˜40% lower in H460 cells, a difference that was significant for cultures in complete or glutamine-free media.

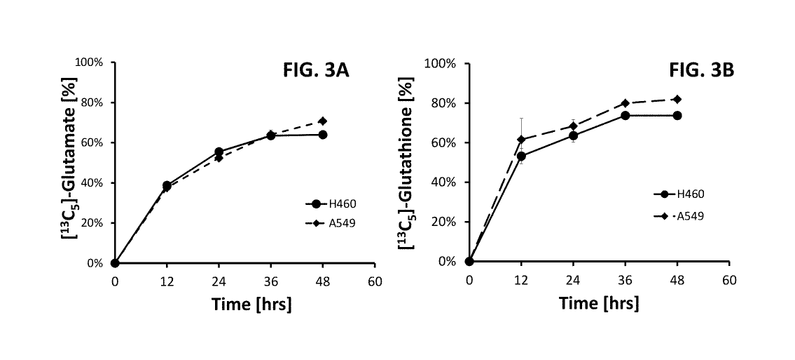

To test the hypothesis that glutathione is in part derived from glutamine, we incubated H460 and A549 cells with glutamine-free medium supplemented with [¹³C₅]-glutamine and monitored ¹³C₅-incorporation into glutamate and glutathione at various time points (FIG. 3). After 12 hrs, excreted glutamate was >38% labeled in both cell lines. Excreted glutathione were 53% and 62% [¹³C₅] labeled for H460 and A549 cells, respectively, indicating that it is derived from the [¹³C₅]-glutamine. The cell-free medium control did not show any ¹³C-labeled glutamate or glutathione. [¹³C₅]-glutamine-supplemented media did not affect cell growth compared to complete media (data not shown).

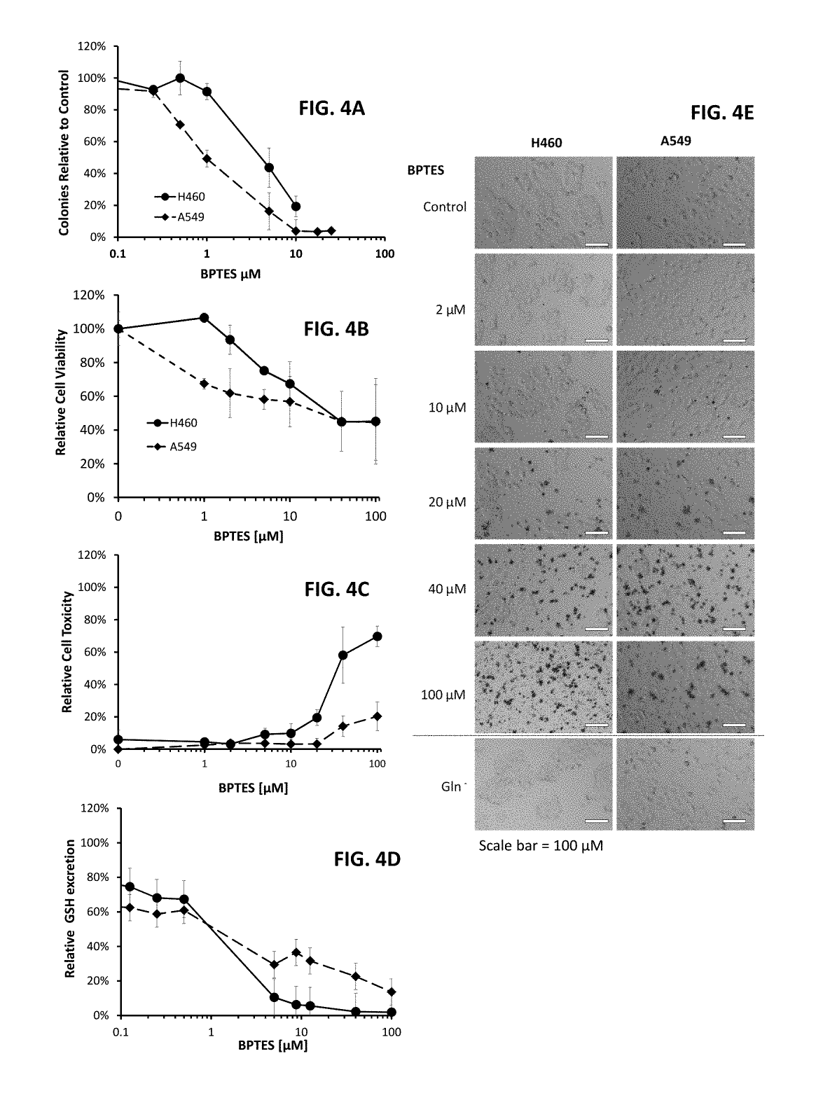

GLS catalyzes the conversion of glutamine to glutamate, a precursor of glutathione synthesis and an essential step in glutamine utilization. To determine the role of glutamine as a pre-cursor for glutathione synthesis, cells were treated with GLS-inhibitor BPTES. BPTES treatment led to significant growth inhibition, toxicity, and abolished glutathione excretion (FIGS. 4A-4C). The effects of GLS inhibition were similar to culturing cells in glutamine-free medium (data not shown). Cell responses to BPTES were assessed by four different assays (CellTiter, CellTiter-Glo, CelITox, colony formation), of which the colony formation showed a dose response with ED₅₀ of 4.2 and 1.0 μM BPTES for H460 and A549, respectively (FIG. 4A). The metabolic response to BPTES showed an ED₅₀ of 7.2 and 4.0 μM BPTES for A549 and H460, respectively. At greater BPTES concentrations formation and excretion of glutathione (FIG. 4D) and the intermediate metabolites glutamate and γ-glutamyl-cysteine were essentially abolished (data not shown). H460 and A549 responses to BPTES treatments demonstrate a visual increase in cell death at concentration of 10, 20, 40, and 100 μM, but no cell death was observed in cells cultured in glutamine-free medium. (FIG. 4F).

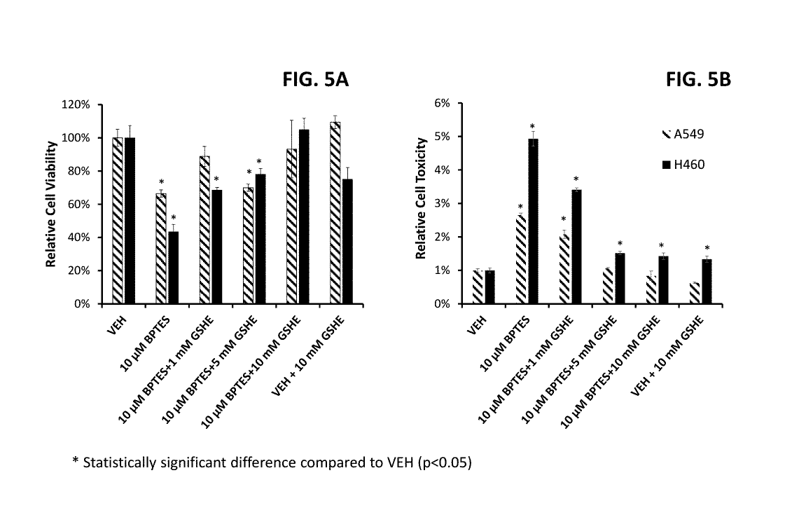

The importance of glutamine-derived GSH for cell viability and against cytotoxicity was investigated using media supplemented with GSHE, the bioavailable form of GSH. Initial treatment with 10 μM BPTES reduced viability and induced cytotoxicity (FIGS. 5A-5B) in both tumor cell lines. Addition of 1-10 mM GSHE showed a dose-dependent protection against BPTES-induced cytotoxicity in both cell lines. As in the previous experiment, the BPTES effects were less pronounced in the A549 compared to the H460.

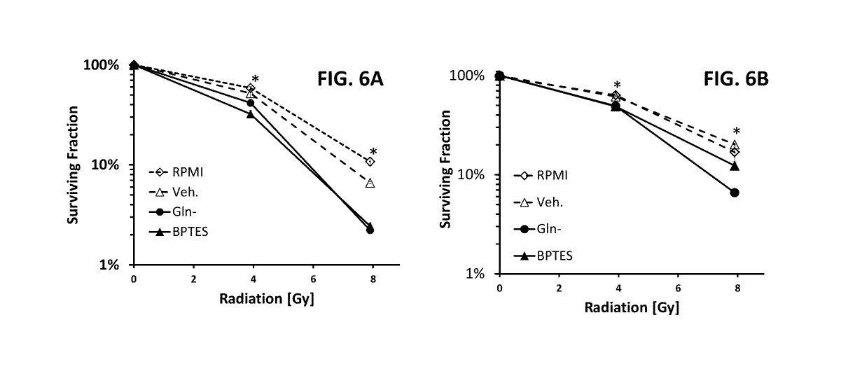

To demonstrate that the glutamine-derived glutathione is biologically, and likely clinically relevant, responses to ionizing radiation was determined in both tumor cell lines after culturing in glutamine-free media or after treatment with BPTES (FIG. 6). The results demonstrate the biological importance of glutamine-derived glutathione in defense against oxidative stress induced by ionizing radiation. First, lack of glutamine significantly increased radiation sensitivity for both A549 and H460 as compared to control at both the 4 Gy and 8 Gy levels (p<0.01 for all) suggesting an essential role for glutamine-derived glutathione in defense against radiation-induced injury (FIGS. 6A-6B). Second, inhibition of GLS by BPTES produced a radiation-induced reduction in colony formation in H460 and A549 cells at 4 Gy and 8 Gy levels as compared to control cells (p<0.001). Sensitization was slightly less pronounced in A549 cells compared to H460 (FIGS. 6A-6B). These results suggests that GLS is intimately linked to GSH production and defense against the oxidative stress created by ionizing radiation exposure, and further implicates glutamine in the response to therapeutics.

DISCUSSION: Glutamine utilization has been shown to promote tumor cell proliferation and GLS activity has been identified as a viable target for cancer therapy. The common belief is that glutamine provides additional building blocks for biosynthesis and energy production. We herein provide strong evidence that glutamine is essential for glutathione production, and therefore contributes directly to cellular defense systems.

Studying the uptake and release of nutrients, it was not surprising to observe greater glutamine uptake and glutamate excretion in the faster growing carcinoma cell lines (H460 and A549) compared to the MRC-5 alveolar fibroblasts (FIGS. 2A and 7A). Under our culture conditions H460 cells consume glutamine and excrete glutamate over the first 48 hrs. After that, glutamine is depleted in H460 cultures, and H460 cells start to utilize glutamate as a carbon source. These results suggest that the initial abundance of glutamine leads to its rapid uptake and conversion to glutamate. However, the intracellular glutamate pool is limited and excessive glutamate is excreted into the medium (FIG. 7B). Adjusting for doubling time, each cell line utilizes about 4 μmoles of glutamine per doubling. For example, H460 reduced glutamine concentration in culture medium from 2 mM to 1.2 mM, which translates to 0.8 μmoles/ml. Therefore, H460 cells cultured in 5 ml media use 4 μmoles (5 ml×0.8 μmoles/ml) in 24 hours corresponding to approximately 2 doubling times.

Surprisingly, both tumor cell lines excreted large amounts of glutathione (FIG. 2B). The H460 and A549 cultures starting with 100,000 cells produced and excreted over the first 48 hrs a minimum of 2.0±1.10 and 0.62±0.09 μmoles of glutathione, respectively. In contrast, MRC-5 fibroblast excreted 0.12±0.00 μmoles during same time period. The amounts of glutathione excreted by 48 hours corresponded to 28.3% and 15.6% of total glutamine utilized by H460 and A549, respectively. The isotope-labeled glutamine experiment indicates that at least 50% of the excreted glutathione is replaced within 12 hrs, demonstrating rapid glutathione turnover. These steady state amounts do not account for any glutathione consumed or utilized for cellular defense during that time period and therefore underestimate the actual amount synthesized and excreted. Together these results emphasize the fact that lung tumors spend huge efforts on glutathione synthesis and excretion. The high glutathione turnover is consistent with the short half-life (2-6 hrs) of glutathione reported in lung tumors, and suggests that glutathione synthesis and cycling in lung tumors and tumor cells is much higher than estimated from steady state measurements.

Correlation analysis clearly suggests a mechanistic link between the glutamine uptake and glutathione excretion (FIG. 2C). Subsequent culturing in glutamine-free media abolished glutathione synthesis and excretion (data not shown) indicating that glutathione synthesis and excretion are tightly linked to, and even dependent on, available glutamine. Culturing in glutamine-free medium significantly reduces cell growth, suggesting that glutathione excretion is important for cell proliferation. Intracellular concentrations of glutathione per mg protein mirrored the observation in culture media, with glutathione being higher in tumor cells compared to MRC-5 fibroblasts, however effects were less pronounced. This was not unexpected since intracellular metabolite concentrations are highly controlled through synthesis and degradation processes, and therefore remain within biologically acceptable ranges. In contrast, the extracellular metabolites corresponding to cellular activity are less stringently regulated, and therefore their concentration ranges are much wider than could be tolerated within the cell.

The first step in glutamine utilization for energy production is its conversion to glutamate by GLS. Culturing in glutamine-free media drastically reduced cell proliferation and might have confounded the results. Therefore, to test whether glutamine as an energy source was essential for cell proliferation and a precursor for glutathione synthesis, cells were treated with a specific GLS inhibitor. The dose-response of BPTES treatment showed decreased cell viability based on the colony formation, CellTiter, CellTiter-Glo®, and CellTox™ assays, while glutathione excretion was essentially abolished at >5 μM BPTES (FIG. 4). Together these results provide evidence for a mechanistic link between GLS activity and glutathione excretion. While the effect of BPTES on cell proliferation has been reported previously the fact that glutathione excretion was affected demonstrates for the first time the essential roles of glutamine and GLS-activity for glutathione synthesis and excretion. GLS knockdown in cervical cancer cells reduce intercellular glutathione in radiation resistant HELA cell to relative levels as normal HELA cells suggesting that GLS and glutathione synthesis are mechanistically linked in other tumors as well.

The ED₅₀ for the metabolic effect (˜>5.0 μM BPTES) was observed at the similar to the ED₅₀ for significant biological response, reduction in cell proliferation suggesting mechanistic link between glutathione excretion and cell viability. Glutathione depletion has been reported previously by inhibition of γ-glutamyl-cysteine ligase using 100 to 10,000 μM buthionine sulfoximine. The presented results suggest that CB-839 and BPTES are at least 1000- and 100-fold, respectively, more effective in depletion of glutathione, making them better candidates for combination therapies.

Glutamate is a common metabolite and can be derived from various sources such as glucose metabolism, protein degradation or amino acid metabolism. Based on the media time curves of glutamate, H460 and A549 cells excrete significant amounts of glutamate, suggesting that these cell lines synthesize or liberate large amounts of glutamate (FIG. 7A). The glutamate excretion and glutamate independence depends on the availability of glutamine as a carbon source.

To determine the source of glutamate that is used for glutathione synthesis, cells were cultured in glutamine-free media supplemented with [¹³C₅]-glutamine. Glutathione derived from the [¹³C₅]-glutamine was distinguished from glutathione that is derived from other sources using mass spectrometry. This experiment provided stunning proof that the majority of glutathione is, in fact, synthesized from glutamine-derived glutamate. After 12 hours, approximately 40% ¹³C-carbon incorporation was observed in the intermediate metabolite glutamate (FIG. 3A). Excreted glutathione contained the >50% ¹³C-carbon backbone (FIG. 3B), confirming the active utilization of the metabolic pathway shown in FIG. 1. The stable isotope tracing experiment clearly demonstrates that glutamine is utilized for glutathione synthesis and excretion. The question of why tumor cells spend so much effort on glutathione synthesis and excretion needs to be further investigated. Further it is interesting to note lung tumor cell seem to use glutamine-derived glutamate for glutathione synthesis.

These findings have wide implications in regard to the suitability of a GLS inhibitors such as CB-839 and BPTES in chemotherapy. Low dose treatments with CB-839, BPTES, and other GLS inhibitors, may be sufficient to significantly reduce glutathione synthesis and thereby increase sensitivity to radiotherapy, thereby making GLS inhibitors a prime component of combination therapies. To test the former, we determined the radiosensitivity of the lung tumor cell lines after culturing in glutamine-free media or after BPTES treatment. Both pretreatments caused glutathione depletion and increased radiosensitivity (FIG. 6). This provides additional evidence of the mechanistic link between glutamine consumption and glutathione synthesis and their importance for cell viability.

Conclusion of in vitro experiments. The inventors demonstrate that a significant amount of extracellular glutathione is directly derived from glutamine. This finding adds yet another important function to the already known glutamine dependence of tumor cells and probably stromal components of solid tumor growth as well. Glutaminolysis has been identified as a suitable target for cancer therapies, and the findings presented herein suggest that a GLS inhibitor leads to glutathione depletion and marked changes in response to radiation treatment. Depleting glutathione in this manner is expected to reduce multi-drug resistance and increase sensitivity to oxidative stress caused by radiation therapy, leading to greater cytotoxicity and tumor response.

### In Vivo Study

Rationale: In our preliminary studies, culturing tumor cells in glutamine-free media or treating with a GLS inhibitor (BPTES and CB-839) led to reduced cell proliferation and viability and abolished glutathione excretion (described above). Our data demonstrated that more than (>) 50% of excreted glutathione is derived from glutamine, and inhibition of GLS markedly radiosensitized the lung tumor cell lines, suggesting an important and previously underappreciated role of glutamine-derived glutathione in the normal cytoprotective state of malignant cells. This finding that glutamine-derived glutathione plays an important role in radiation sensitivity of tumor cell adds yet another important function to the already known glutamine dependence of tumor cells and probably tumor tissue and microenvironment as well. Therefore, the goals for this following in vivo experiments were to translate our in vitro observations to a pre-clinical mouse xenograft mold to demonstrate the importance of glutamine-derive glutathione to protect lung tumors against radiation induce injury and killing and to show that treatment with GLS-inhibitors increases response to radiation therapy.

METHOD: Mouse xenograft experiments. For the first in vivo study, H460 cell-derived tumors were engrafted on the hind flank of 20 male. As tumors reached a size of >100 mm³ (day #12), mice were divided into four groups; (1) control (2) CB-839, (3) 18 Gy radiation and (4) CB-839 plus 18 Gy radiation. For the second in vivo experiment, H460 cell-derived tumors were engrafted on the hind flank of 50 female nude mice. As tumors reached a size of >100 mm³, mice were divided into 10 groups; (1) Vehicle only, (2) 2 Gy radiation, (3) 4 Gy radiation, (4) 8 Gy radiation, (5) 12 Gy radiation, (6) Vehicle only plus CB-839, (7) 2 Gy radiation plus CB-839, (8) 4 Gy radiation plus CB-839, (9) Gy radiation plus CB-839, (10) 12 Gy radiation plus CB-839.

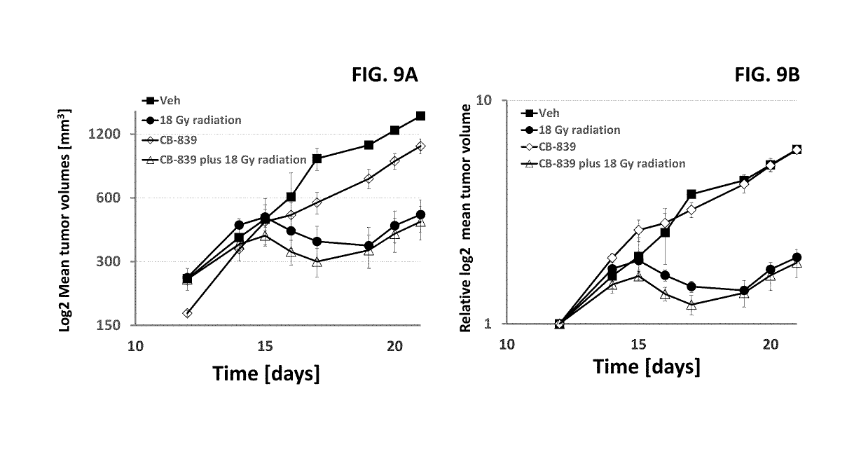

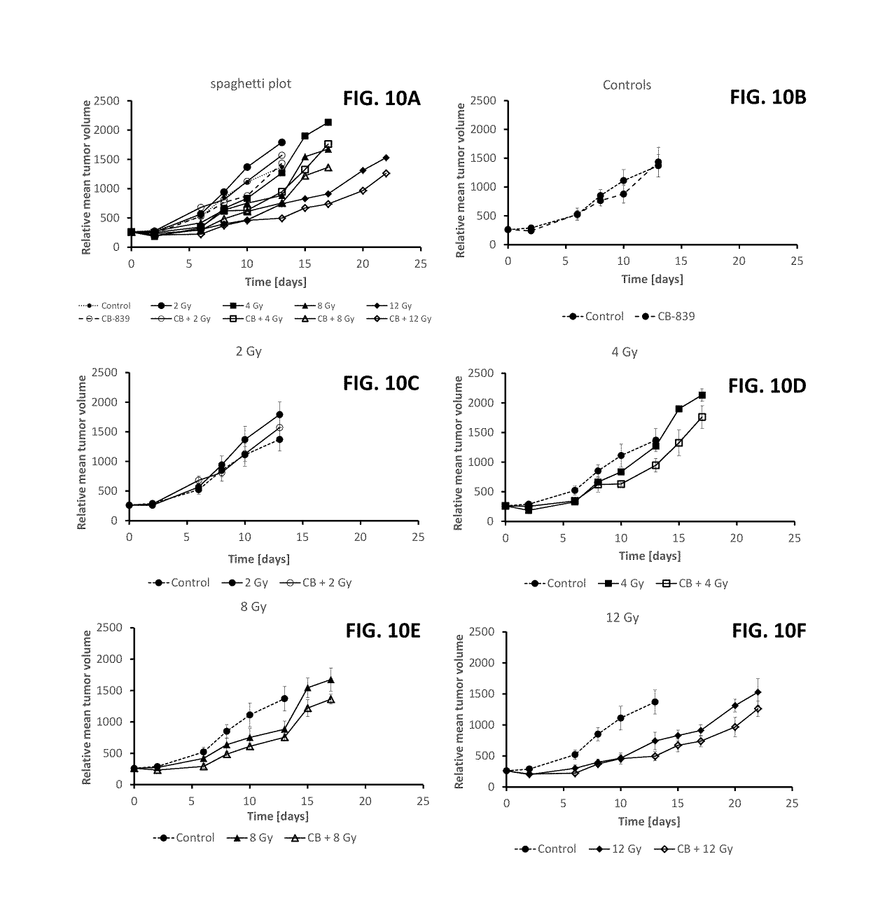

CB-839 was given three times at 20 mg/kg by oral gavage at 28, 16 and 4 hrs prior to a single dose of 18 Gy radiation. CB-839 was administered by oral gavage as solution of 200 mg/kg CB-839 in 200 ul 25% (w/v) hydroxypropyl-b-cyclodextrin in 10 mmol/Lcitrate, pH 2 or 25% (w/v) hydroxypropyl- b-cyclodextrin in 10 mmol/L citrate, pH 2. Thirty minutes prior to radiation treatment, 50 μl blood was taken form tail vein and serum was analyzed for reduced and oxidized GSH by LC-MS. Radiation was given as a single 1Gy/min dose for 2, 4, 8, 12, or 18 min respectively. Tumor volume measurements were repeated at the indicated days until termination of the animals. Tumor size was recorded as largest lateral (indicated in the tables by “a”) and collateral (indicated in the tables by “b”) measurements. Tumor volume was computed by multiplying “a” times (“a” X “b”)/2. The “Mean tumor volumes” and “Relative tumor volume” for male and female mice are graphed in FIGS. 9A-B and FIGS. 10A-F, respectively.

RESULTS: Relevant preliminary data—Effect of GLS inhibitor, CB-839, on response to radiation and serum glutathione: The inventors established H460 cells-derived tumors on the hind flank of 20 male nude mice. As tumors reached a size of >100 mm³ (day #12) mice were divided into four groups; (1) vehicle (2) CB-839, (3) radiation and (4) CB-839 plus radiation. CB-839 was given at 20 mg/kg by oral gavage at 28, 16 and 4 hrs prior to a single dose of 18 Gy radiation. Tumor growth was further monitored for 5 days. At day #17 mean tumor size of mice receiving CB-839 plus radiation were 20% and 90% smaller compared to radiation or CB-839 alone, respectively (FIG. 9A). The three CB-839 treatments did not affect subsequent tumor growth compared to control. At day #12 prior to radiation treatment, blood was taken from tail vein and serum was analyzed for reduced and oxidized GSH by LC-MS. Total GSH was 56% lower in mice treated with CB-839 compared to none treated mice (FIG. 9B). Together these results show that CB-839, a novel GLS inhibitor, leads to GSH depletion and increases sensitivity to radiation therapy. The radiation treatment seems to account for most of the effects.

To further demonstrate suitability of GLS-inhibitors to improve response to radiation therapy, the inventors conducted a second mouse xenograft study in female nude mice that were treated with 3 doses (orally every 12 hrs) of CB-839 and various doses of radiation. Various doses of radiation were chosen because in the first experiment most of the effect was attributed to the radiation and, in clinical practice, low radiation dose is expected to limit side effects. For this in vivo experiment, H460 cell-derived tumors were engrafted on the hind flank of 50 female nude mice. As tumors reached a size of >100 mm³, the mice were randomly assigned to following groups of 5 mice each: (1) Vehicle (2) 2 Gy radiation (3) 4 Gy radiation, (4) 8 Gy radiation (5) 12 Gy radiation (6) CB-839 only, (7) CB-839 plus 2 Gy radiation (8) CB-839 plus 4 Gy radiation (9) CB-839 plus 8 Gy radiation (10) CB-839 plus 12 Gy radiation. CB-839 was given by gavage 28 hrs, 16 hrs and 4 hrs prior to radiation. FIG. 10A shows the tumor growth cure for all groups. As described above with the male mice, CB-839 treatment alone did not affect the tumor growth (FIG. 10B). The addition of 2 Gy radiation alone caused a repopulation-related acceleration of tumor growth by 23% and 31% after 10 and 13 days compared to control. The slightly accelerated tumor growth by 2 Gy radiation, was prevented by CB-839 treatment (FIG. 10C). Higher radiation doses reduced tumor growth and in combination with CB-839 treatment, tumor growth was further reduced by 15 to 30% depending on combination of drug to radiation and day of measurement (FIG. 10D-10F). At the time of radiation treatment, total glutathione in blood were 53% lower. At that time, mice had received three doses of 200 mg/Kg/bw CB-839 at 28 hrs, 16 hrs and 4 hrs earlier.

Conclusion of in vivo experiments. The experiments described above consistently show that combining radiation therapy with a short course of orally administered GLS-inhibitor, CB-839, resulted in reduced tumor growth. This is true for both male and female mice and various combinations of radiation doses plus GLS-inhibitor. In both studies, the GLS-inhibitor led to lower serum glutathione (reduced or oxidized). This is in agreement with the proposed mechanism by which GLS-inhibitors sensitizes tumors to radiation therapy.

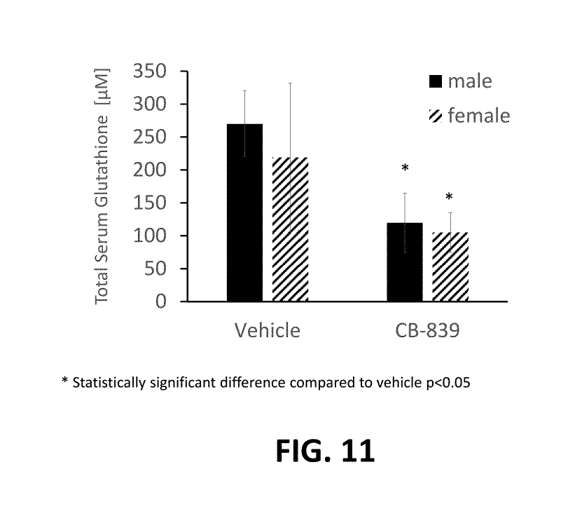

While the present invention has been described with reference to CB-839 and BPTES, the full scope of the present invention is not limited to those two GLS-inhibitors. In all the experiments GLS-inhibitors depleted glutathione in vitro (FIG. 4D) and reduction systemic glutathione in vivo (FIG. 11). Therefore, any GLS inhibitor will cause reduction and depletion for glutathione, an antioxidant important to protect tumor cells against damage from ionizing radiation. The effective amounts of GLS inhibitors are well-known to a person of ordinary skill in the art. In addition, the effective dosage of radiation is well-known for numerous types of cancer.

Clinical Application. Clinical implementation of the GLS-inhibitors is preferably as follows. In the clinical setting, the serum glutathione changes after dosing can be monitored and the inventors have shown that the effect of a single fraction of radiation is improved by >20% when the serum glutathione drops by >50%. Thus, the dose and delivery strategy in each patient can be determined that reduces glutathione by >50% in the blood stream. At that time, the patient would be approved to start radiation. This is a ‘personalized medicine’ approach that is expected to work very well with the tools already available in clinical practice.

### REFERENCES

Boysen, Gunnar. 2017. “The Glutathione Conundrum: Stoichiometric Disconnect between Its Formation and Oxidative Stress.” Chemical Research in Toxicology 30(5):1113-16.
Franken, Nicolaas A. P., Hans M. Rodermond, Jan Stap, Jaap Haveman, and Chris van Bree. 2006. “Clonogenic Assay of Cells in Vitro.” Nature Protocols 1(5):2315-19. Retrieved (http://www.ncbi.nlm.nih.gov/pubmed/17406473).
Vander Heiden, Matthew G., Lewis C. Cantley, and Craig B. Thompson. 2009. “Understanding the Warburg Effect: The Metabolic Requirements of Cell Proliferation.” Science (New York, N.Y.) 324(5930):1029-33. Retrieved (http://www.ncbi.nlm.nih.gov/pubmed/19460998).
Sappington, Daniel R. et al. 2016. “Glutamine Drives Glutathione Synthesis and Contributes to Radiation Sensitivity of A549 and H460 Lung Cancer Cell Lines.” Biochimica et Biophysica Acta—General Subjects 1860(4):836-43.
Sappington, Daniel R. et al. 2017. “Diagnosis of Lung Tumor Types Based on Metabolomic Profiles in Lymph Node Aspirates.” Cancer Treatment and Reseach Communication in Press.
Shukla, Krupa et al. 2012. “Design, Synthesis, and Pharmacological Evaluation of Bis-2-(5-Phenylacetamido-1,2,4-Thiadiazol-2-Yl)ethyl Sulfide 3 (BPTES) Analogs as Glutaminase Inhibitors.” Journal of Medicinal Chemistry 55(23):10551-63. Retrieved (http://www.ncbi.nlm.nih.gov/pubmed/23151085).

The present invention has been described with reference to certain preferred and alternative embodiments that are intended to be exemplary only and not limiting to the full scope of the present invention.

We claim:

1. A method of increasing radiation sensitivity of tumor cells in a subject, said method comprising the steps of:
(a) administering to said subject an effective amount of a glutaminase inhibitor; and
(b) exposing said tumor cells to radiation.

2. The method of claim 1, wherein said glutaminase inhibitor is CB-839.

3. The method of claim 1, wherein said glutaminase inhibitor is BPTES.

4. The method of claim 1, wherein step (a) is repeated prior to step (b).

5. A method of treating a tumor in a subject, said method comprising the steps of:
(a) administering to said subject an effective amount of a glutaminase inhibitor;
(b) exposing said tumor to radiation in a dose effective to reduce a size of said tumor.

6. The method of claim 5, wherein said glutaminase inhibitor is CB-839.

7. The method of claim 5, wherein said glutaminase inhibitor is BPTES.

8. The method of claim 5, wherein step (a) is repeated prior to step (b).
